(19) United States
(12) Patent Application Publication
(10) Pub. No.: US 2013/0018094 A1
(43) Pub. Date: Jan. 17, 2013

(54) Prophylactic or Therapeutic Agent For Gum Disease or Apical Periodontitis

(71) Applicants: Nobuyuki Kawashima, Tokyo (JP); Noriyuki Suzuki, Tokyo (JP)

(72) Inventors: Nobuyuki Kawashima, Tokyo (JP); Noriyuki Suzuki, Tokyo (JP)

(21) Appl. No.: 13/497,932

(22) PCT Filed: Sep. 21, 2010
(86) PCT No.: PCT/JP2010/066285

§ 371 Date: Sep. 17, 2012

(30) Foreign Application Priority Data
Sep. 24, 2009 (JP) 2009-218590

(51) Int. Cl. A61K 31/336; A61P 1/02

(52) U.S. Cl. 514/475

(57) ABSTRACT

Monosodium (2S,3S)-3-[[(1S)-1-isobutozymethyl-3-methylbutyl]-carbamoyl]oxirane-2-carboxylate represented by the following formula is used as a prophylactic or therapeutic agent for periodontal diseases including gum disease and apical periodontitis.

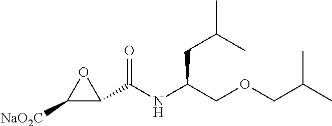

### TECHNICAL FIELD

The present invention relates to a prophylactic or therapeutic agent for gum disease or apical periodontitis comprising as an active ingredient an epoxysuccinate derivative or physiologically acceptable salt thereof.

### BACKGROUND ART

Periodontal diseases can be broadly classified into gum disease, in which inflammation occurs surrounding the tooth (gingivitis and periodontitis), and apical periodontitis, in which inflammation occurs in the apical area of the tooth. The state in which periodontal pathogenic bacteria have deposited as plaque surrounding the tooth and caused inflammation localized only to the gingiva is called gingivitis, and the state in which inflammation has also spread to the periodontal membrane and resorption of the alveolar bone is seen surrounding the tooth is called periodontitis. Furthermore, secondary caries and inflammation of the dental pulp (pulpitis) are caused by caries pathogenic bacteria, but as these progress, bacterial flora is formed in the gaps (pulp cavities) in which the pulp has necrotized. The inflammatory response induced by this in the apical area is called apical periodontitis. Necrosis of the alveolar bone on the apical periphery is also seen in apical periodontitis. That is, both periodontitis and apical periodontitis are part of the body's preventative response to bacterial infiltration, where in the former, the major characteristic is that infiltration of inflammatory cells is seen in the marginal periodontal tissue, and in the latter, infiltration of inflammatory cells is seen in the apical periodontal region and resorption of the alveolar bone in that area is also seen. The alveolar bone present surrounding the tooth plays an important role in preserving the tooth and allowing it to function, and resorption of this bone causes tooth loss. That is, periodontitis and apical periodontitis can be considered major factors in tooth loss. Chewing with one's own teeth is very important for maintain or improving one's quality of life, and in this sense as well, sound methods of treatment of periodontitis and apical periodontitis are anticipated.

Incidentally, Patent Reference 1 states that peptidyl hydroxamic acid derivatives can be used as prophylactic or therapeutic agents for periodontal tissue destruction and are useful in apical periodontitis.

Furthermore, Patent Reference 2 states that methane bisphosphonic acid derivatives or hydrates thereof have the action of suppressing cell infiltration into the affected area in periodontal diseases and are useful in the prevention and treatment of periodontal diseases such as gum disease and apical periodontitis.

Additionally, Patent Reference 3 discusses applications of leucine amide derivatives, which are cathepsin cysteine protease inhibitors, in gum disease.

Meanwhile, it is known that monosodium (2S,3S)-3-[[(1S)-1-isobutozymethyl-3-methylbutyl]-carbamoyl]oxirane-2-carboxylate (also abbreviated as compound A hereinafter) represented by the following formula:

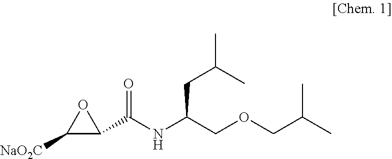

has a cathepsin K inhibiting action and is useful as a therapeutic agent for chronic rheumatism, osteoporosis and so forth (Patent Reference 4).

As of now there have been no reports that the compounds stated in Patent References 1 through 3 have been used clinically as prophylactic or therapeutic agents for gum disease or apical periodontitis, and there is no mention in these references that compound A is useful in gum disease or apical periodontitis.

### PRIOR ART REFERENCES

### Patent References

Patent Reference 1: JP-A-08-283177
Patent Reference 2: WO 01/005403
Patent Reference 3: WO 2006/056047
Patent Reference 4: WO 99/11640

### SUMMARY OF THE INVENTION

### Problems to be Solved by the Invention

The objective of the present invention is to provide a prophylactic or therapeutic agent for gum disease or apical periodontitis comprising as an active ingredient an epoxysuccinate derivative or physiologically acceptable salt thereof.

### Means for Solving Problems

The present invention relates to a prophylactic or therapeutic agent for periodontal diseases comprising as an active ingredient an epoxysuccinate derivative represented by general formula (I) or physiologically acceptable salt thereof:

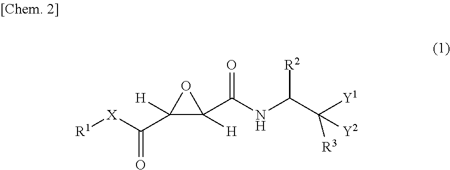

(wherein R¹ represents a hydrogen atom, alkyl group having 1-10 carbon atoms, alkenyl group having 2-10 carbon atoms, alkynyl group having 2-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;

R² represents an alkyl group having 1-10 carbon atoms, alkenyl group having 2-10 carbon atoms, alkynyl group having 2-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
R³ represents a hydrogen atom, alkyl group having 1-10 carbon atoms, alkenyl group having 2-10 carbon atoms, alkynyl group having 2-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
X represents —O— or NR⁴—, wherein R⁴ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
Y¹ represents OR⁵, SR⁶ or NR⁷R⁸, wherein R⁵ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, acyl group having 2-20 carbon atoms, heterocyclic group having 3-12 carbon atoms, or heterocyclic alkyl group made up of a heterocyclic group having 3-12 carbon atoms and an alkyl group having 1-6 carbon atoms; R⁶ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, acyl group having 2-20 carbon atoms, heterocyclic group having 3-12 carbon atoms, or heterocyclic alkyl group made up of a heterocyclic group having 3-12 carbon atoms and an alkyl group having 1-6 carbon atoms; R⁷ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, acyl group having 2-20 carbon atoms, heterocyclic group having 3-12 carbon atoms, or heterocyclic alkyl group made up of a heterocyclic group having 3-12 carbon atoms and an alkyl group having 1-6 carbon atoms; and R⁸ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms; and
Y² represents a hydrogen atom or alkyl group having 1-10 carbon atoms, or Y¹ and Y² together represent ═O, ═S, ═N—R⁹ or ═N—OR¹⁰, wherein R⁹ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms; and R¹⁹ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
wherein any of the alkyl groups of the aforementioned R⁵ through R¹⁹ may have one or two or more substituents selected from the set made up of a hydroxyl group, amino group, alkylamino group having 1-6 carbon atoms, dialkylamino group having a total of 2-12 carbon atoms, alkoxy group having 1-6 carbon atoms, carboxyl group, alkoxycarbonyl group having 2-7 carbon atoms, carbamoyl group, alkylaminocarbonyl group having 2-7 carbon atoms, dialkylaminocarbonyl group having a total of 3-13 carbon atoms, and guanidino group; furthermore, each aryl group and each heterocyclic group of the aforementioned R¹ through R¹⁹ may have one or two or more substituents selected from the set made up of an alkyl group having 1-6 carbon atoms, hydroxyl group, amino group, alkylamino group having 1-6 carbon atoms, dialkylamino group having a total of 2-12 carbon atoms, alkoxy group having 1-6 carbon atoms, halogen atom, haloalkyl group having 1-6 carbon atoms, cyano group, nitro group, carboxyl group, alkoxycarbonyl group having 2-7 carbon atoms, carbamoyl group, alkylaminocarbonyl group having 2-7 carbon atoms, dialkylaminocarbonyl group having a total of 3-13 carbon atoms, amidino group, and guanidino group).

Furthermore, the present invention relates to a prophylactic or therapeutic agent for gum disease comprising as an active ingredient an epoxysuccinate derivative represented by general formula (I) or physiologically acceptable salt thereof.

Additionally, the present invention relates to a prophylactic or therapeutic agent for apical periodontitis comprising as an active ingredient an epoxysuccinate derivative represented by general formula (I) or physiologically acceptable salt thereof.

### Advantageous Effect of the Invention

The medicinal composition comprising as an active ingredient an epoxysuccinate derivative represented by general formula (I) or physiologically acceptable salt thereof is expected to have an excellent prophylactic or therapeutic effect on gum disease or apical periodontitis.

### BRIEF DESCRIPTION OF THE DRAWINGS

FIG. 1 is a micro-CT of a control group.

FIG. 2 is a micro-CT of a compound A administration group.

FIG. 3 is the level of expression of IL-1α of the control group and the compound A administration group.

FIG. 4 is the level of expression of IL-6 of the control group and the compound A administration group.

### EMBODIMENTS

The present invention will be described in detail below.

The epoxysuccinate derivative expressed by general formula (I) or physiologically acceptable salt thereof, which is the active ingredient, is a known substance, as mentioned in Patent Reference 4, for example.

Also, the epoxysuccinate derivative expressed by general formula (I) or physiologically acceptable salt thereof can be produced while referring to the methods stated in Patent Reference 4, WO 2004/24672, WO 2004/96785 and so forth.

Preferred compounds of the epoxysuccinate derivative expressed by general formula (I) or physiologically acceptable salt thereof are as follows.

(1) The epoxysuccinate derivative expressed by general formula (I) or physiologically acceptable salt thereof, wherein R′ is a hydrogen atom or alkyl group having 1-6 carbon atoms.

(2) The epoxysuccinate derivative expressed by general formula (I) or stated in item (1) above or physiologically acceptable salt thereof, wherein R² is an alkyl group having 1-6 carbon atoms, phenyl group, or benzyl group.

(3) The epoxysuccinate derivative expressed by general formula (I) or stated in item (1) or (2) above or physiologically acceptable salt thereof, wherein R³ is a hydrogen atom or aryl group having 6-20 carbon atoms.

(4) The epoxysuccinate derivative expressed by general formula (I) or stated in item (1) through (3) above or physiologically acceptable salt thereof, wherein X is —O—.

(5) The epoxysuccinate derivative expressed by general formula (I) or stated in item (1) through (4) above or physiologically acceptable salt thereof, wherein Y′ is a hydroxyl group, alkoxy group having 1-6 carbon atoms, acetoxy group, or aralkyloxy group made up of an aryl group having 6-20 carbon atoms and an alkyl group having 1-6 carbon atoms.

(6) The epoxysuccinate derivative expressed by general formula (I) or physiologically acceptable salt thereof, wherein R² is an isobutyl group or isopropyl group, R³ is a hydrogen atom, Y¹ is OR⁵, and Y² is a hydrogen atom.

(7) The epoxysuccinate derivative expressed by general formula (I) or physiologically acceptable salt thereof, wherein R² is an isobutyl group or isopropyl group, R³ is an aryl group having 6-20 carbon atoms, Y¹ is OR⁵, and Y² is a hydrogen atom.

(8) The epoxysuccinate derivative expressed by general formula (I) or physiologically acceptable salt thereof, wherein Y¹ and Y² together form ═O.

(9) The epoxysuccinate derivative expressed by general formula (I) or stated in item (1) through (8) above or physiologically acceptable salt thereof, wherein the physiologically acceptable salt is an alkali metal salt.

(10) Monosodium (2S,3S)-3-[[(1S)-1-isobutozymethyl-3-methylbutyl]-carbamoyl]oxirane-2-carboxylate.

Pharmacological tests will be described below.

Using a rat apical periodontitis model, pharmacological tests were conducted for monosodium (2S,3S)-3-[[(1S)-1-isobutozymethyl-3-methylbutyl]-carbamoyl]oxirane-2-carboxylate.

### Example 1

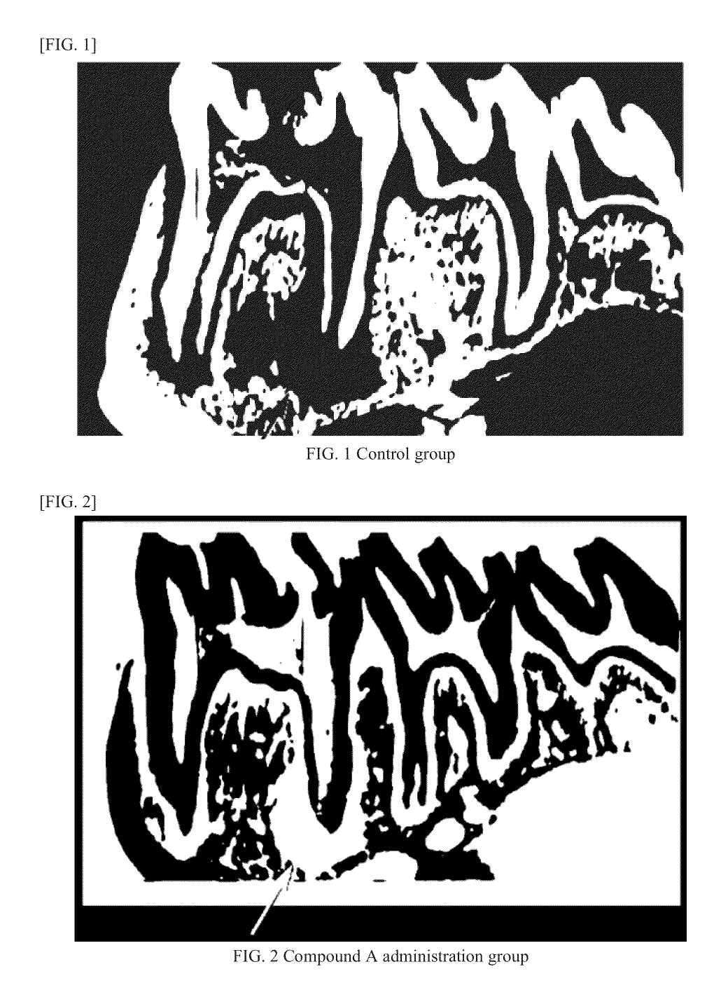

As a result of evaluating the size of apical lesions by micro-CT, it was seen that smaller apical lesions were formed in the compound A administration group than in the control group (FIGS. 1, 2).

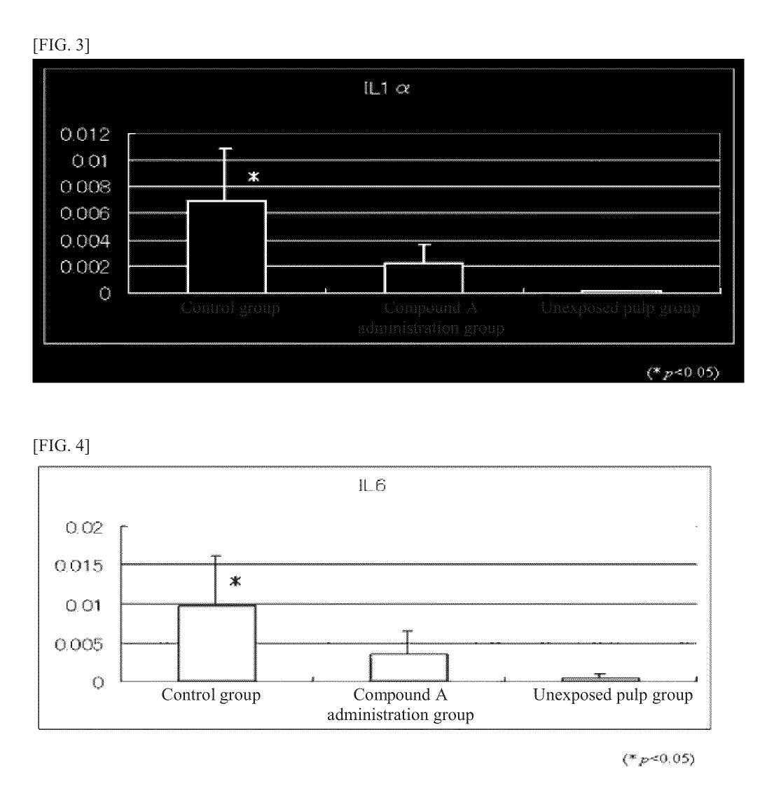

Also, when the levels of expression of inflammatory cytokines in the lesions were compared, it was seen that the levels of expression of IL-1α and IL-6 were significantly suppressed in the compound A administration group compared to the control group (FIGS. 3, 4).

Therefore, the use of the epoxysuccinate derivative represented by general formula (I) or physiologically acceptable salt thereof as a prophylactic or therapeutic agent for periodontal diseases and its use as a prophylactic or therapeutic agent for periodontal tissue destruction are expected, and preferably, it is useful as a prophylactic or therapeutic agent for gum disease and apical periodontitis.

The epoxysuccinate derivative represented by general formula (I) or physiologically acceptable salt thereof may be administered to humans via an appropriate route of administration such as oral or parenteral administration. Note that it can also be used in animals such as dogs and cats suffering from gum disease or apical periodontitis.

Additionally, it can be used in combination with antibiotics, pain killers and anti-inflammatories.

Formulations may be produced in the form of tablets, granules, powders, capsules, suspensions, injectables, dental ointments, buccals and suppositories by ordinary methods used in the field of formulation.

In the preparation of these formulations, in the case of tablets, for example, ordinary excipients, disintegration agents, binders, lubricants, dyes and so forth may be used. Here, examples of excipients include lactose, D-mannitol, crystalline cellulose and dextrose. Examples of disintegration agents include starch and carboxymethyl cellulose calcium (CMC-Ca), and examples of lubricants include magnesium stearate and talc. Examples of binders include hydroxypropyl cellulose (HPC), gelatin and polyvinylpyrrolidone (PVP). In the preparation of injectables, for example, stabilizers, dissolution aids, suspension agents, emulsifiers, soothing agents, relaxants, preservatives and so forth may be used.

The dosage in healthy adults is approximately 0.01 mg to 100 mg per day of the active ingredient epoxysuccinate derivative represented by general formula (I) or physiologically acceptable salt thereof as an injectable, and 1 mg to 2000 mg per day by oral administration, but it can be varied depending on age, health conditions and so forth.

The present invention is described in further detail by citing an example below, but the present invention is not limited thereto.

### Example 1

### Experimental Methods

An experiment was conducted using six-week-old male Wistar rats. After the pulp of the mandibular first molar was exposed using a round bar, apical periodontitis was induced by extracting the pulp and opening it inside the oral cavity. In the experimental groups, monosodium (2S,3S)-3-[[(1S)-1-isobutozymethyl-3-methylbutyl]-carbamoyl]oxirane-2-carboxylate (compound A: 150 mg/kg) was orally administered twice per day. A non-administration group was used as a control. After 21 days, the rats were euthanized and the jawbone surrounding each mandibular molar was extracted. The left side was fixed in 4% paraformaldehyde/phosphate buffer solution (PBS) (4° C., 12 hours), and then the size of the apical lesion was measured by micro-CT imaging (Shimadzu SMX-90CT). After that, it was decalcified with 15% EDTA solution and embedded in OCT compound, and frozen slices 7 μm thick were made. They were stained using hematoxylin eosin (HE) and tartrate-resistant acid phosphatase (TRAP), and examined histologically. On the right side, the lesion portion surrounding the apex was extracted as a lump together with the apex, and after RNA extraction (Qiagen: RNeasy Lipid Tissue Mini Kit), cDNA was created by ordinary methods (Invitrogen: Superscript III), and the levels of expression of inflammatory cytokines (IL-1α and IL-6) were measured by real-time PCR (BioRad: DNA Engine Opticon 2; Invitrogen: Platinum SYBR Green qPCR SuperMix).

(Results)

As a result of evaluating the size of apical lesions by micro-CT, it was seen that larger apical lesions were formed in the control group. On the other hand, although apical lesions were formed even in the compound A administration group, their size was smaller than in the control group (FIGS. 1, 2). Furthermore, as a result of histological examination, numerous osteoclasts were observed on the alveolar bone surrounding the apex, and an image of vigorous bone resorption was seen. In contrast, in the compound A administration group, although osteoclasts were seen, their number tended to be small. When expression of inflammatory cytokines in the lesions was compared, it was seen that expression of IL-1α and IL-6 was significantly suppressed in the compound A administration group (FIGS. 3, 4).

As is clear from FIGS. 1 through 4, compound A exhibited an excellent suppressive effect against progression of rat experimental apical periodontitis.

What is claimed is:

1. A method for the treatment of periodontal diseases, the method comprising administering to a subject in need thereof a therapeutically effective amount of an epoxysuccinate derivative represented by general formula (I) or physiologically acceptable salt thereof: 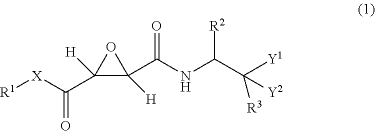
(wherein R¹ represents a hydrogen atom, alkyl group having 1-10 carbon atoms, alkenyl group having 2-10 carbon atoms, alkynyl group having 2-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
R² represents an alkyl group having 1-10 carbon atoms, alkenyl group having 2-10 carbon atoms, alkynyl group having 2-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
R³ represents a hydrogen atom, alkyl group having 1-10 carbon atoms, alkenyl group having 2-10 carbon atoms, alkynyl group having 2-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
X represents —O— or NR⁴—, wherein R⁴ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
Y¹ represents OR⁵, SR⁶ or NR⁷R⁸, wherein R⁵ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, acyl group having 2-20 carbon atoms, heterocyclic group having 3-12 carbon atoms, or heterocyclic alkyl group made up of a heterocyclic group having 3-12 carbon atoms and an alkyl group having 1-6 carbon atoms; R⁶ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, acyl group having 2-20 carbon atoms, heterocyclic group having 3-12 carbon atoms, or heterocyclic alkyl group made up of a heterocyclic group having 3-12 carbon atoms and an alkyl group having 1-6 carbon atoms; R⁷ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, acyl group having 2-20 carbon atoms, heterocyclic group having 3-12 carbon atoms, or heterocyclic alkyl group made up of a heterocyclic group having 3-12 carbon atoms and an alkyl group having 1-6 carbon atoms; and R⁸ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms; and
Y² represents a hydrogen atom or alkyl group having 1-10 carbon atoms, or Y¹ and Y² together represent ═O, ═S, ═N—R⁹ or ═N—OR¹⁰, wherein R⁹ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms; and R¹⁰ represents a hydrogen atom, alkyl group having 1-10 carbon atoms, aryl group having 6-20 carbon atoms, aralkyl group made up of an aryl group having 6-20 carbon atoms and an alkyl group having 1-6 carbon atoms, heterocyclic group having 3-12 carbon atoms, or heterocyclic alkyl group made up of a heterocyclic group having 3-12 carbon atoms and an alkyl group having 1-6 carbon atoms;
wherein any of the alkyl groups of the aforementioned R⁵ through R¹⁰ may have one or two or more substituents selected from the set made up of a hydroxyl group, amino group, alkylamino group having 1-6 carbon atoms, dialkylamino group having a total of 2-12 carbon atoms, alkoxy group having 1-6 carbon atoms, carboxyl group, alkoxycarbonyl group having 2-7 carbon atoms, carbamoyl group, alkylaminocarbonyl group having 2-7 carbon atoms, dialkylaminocarbonyl group having a total of 3-13 carbon atoms, and guanidino group; furthermore, each aryl group and each heterocyclic group of the aforementioned R¹ through R¹³ may have one or two or more substituents selected from the set made up of an alkyl group having 1-6 carbon atoms, hydroxyl group, amino group, alkylamino group having 1-6 carbon atoms, dialkylamino group having a total of 2-12 carbon atoms, alkoxy group having 1-6 carbon atoms, halogen atom, haloalkyl group having 1-6 carbon atoms, cyano group, nitro group, carboxyl group, alkoxycarbonyl group having 2-7 carbon atoms, carbamoyl group, alkylaminocarbonyl group having 2-7 carbon atoms, dialkylaminocarbonyl group having a total of 3-13 carbon atoms, amidino group, and guanidino group).

2. The method according to claim 1, wherein R¹ is a hydrogen atom or alkyl group having 1-6 carbon atoms.

3. The method according to claim 1, wherein R² is an alkyl group having 1-6 carbon atoms, phenyl group, or benzyl group.

4. The method according to claim 1, wherein R³ is a hydrogen atom or aryl group having 6-20 carbon atoms.

5. The method according to claim 1, wherein X is —O—.

6. The method according to claim 1, wherein Y¹ is a hydroxyl group, alkoxy group having 1-6 carbon atoms, acetoxy group, or aralkyloxy group made up of an aryl group having 6-20 carbon atoms and an alkyl group having 1-6 carbon atoms.

7. The method according to claim 1, wherein R² is an isobutyl group or isopropyl group, R³ is a hydrogen atom, Y¹ is OR⁵, and Y² is a hydrogen atom.

8. The method according to claim 1, wherein R² is an isobutyl group or isopropyl group, R³ is an aryl group having 6-20 carbon atoms, Y¹ is OR⁵, and Y² is a hydrogen atom.

9. The method according to claim 1, wherein Y¹ and Y² together form ═O.

10. The method according to claim 1, wherein the physiologically acceptable salt is an alkali metal salt.

11. A method for the treatment of periodontal diseases comprising administering a therapeutically effective amount of monosodium (2S,3S)-3-[[(1S)-1-isobutozymethyl-3-methylbutyl]-carbamoyl]oxirane-2-carboxylate to a subject in need thereof.

12. The method according to claim 11, wherein the periodontal disease is gum disease.

13. The method according to claim 11, wherein the periodontal disease is apical periodontitis.
